(19) United States
(12) Patent Application Publication
(10) Pub. No.: US 2011/0236911 A1
(43) Pub. Date: Sep. 29, 2011

(54) Precipitating Substrate for Bio-Layer Interferometry

(75) Inventors: Sae Hyun Choo, Sunnyvale, CA (US); Weilei Ma, Mountain View, CA (US); Jing Wei, Sunnyvale, CA (US)

(73) Assignee: FORTEBIO, INC., Menlo Park, CA (US)

(21) Appl. No.: 13/052,001

(22) Filed: Mar. 18, 2011

Related U.S. Application Data
(60) Provisional application No. 61/315,386, filed on Mar. 18, 2010.

(51) Int. Cl. G01N 33/53; C12M 1/34

(52) U.S. Cl. 435/7.91; 435/287.2

(57) ABSTRACT

Improved apparatus, compositions, and methods for carrying out interferometry-based assays for detecting analytes in a sample through the use of a precipitating substrate to enhance an interferometry binding signal, and kits useful for carrying out these assays. Methods comprise providing an optical assembly comprising an optical element with a transparent material and adapted for coupling to a light source via a fiber, a first reflective surface and a second reflecting surface having a first analyte-binding molecule and separated from said first surface by a distance, d, exposing said optical element to a sample comprising said analyte, a second analyte binding molecule, and a precipitating substrate; and detecting a change in thickness at said first reflective surface thereby detecting said analyte in said sample. Kits include reagents for derivatizing assay components along with packaging and instructions for use.

### CROSS REFERENCE TO RELATED APPLICATIONS

This application claims the benefit of U.S. provisional application 61/315,386 filed Mar. 18, 2010 which is incorporated herein by reference in its entirety for all purposes.

This application is related to co-owned U.S. Pat. Nos. 5,804,453 issued Sep. 8, 1998; 7,319,525 issued Jan. 15, 2008; 7,394,547 issued Jul. 1, 2008; 7,445,887 issued Nov. 4, 2008; 7,656,536 issued Feb. 2, 2010 and 7,728,982 issued Jun. 1, 2010, all of which are hereby incorporated by reference in their entirety for all purposes.

### BACKGROUND OF THE INVENTION

1. Field of the Invention

The present invention relates to methods, articles of manufacture, and kits directed to precipitating substrate sensors.

2. Description of the Related Art

Diagnostic tests based on a binding event between members of an analyte-anti-analyte binding pair are widely used in medical, veterinary, agricultural and research applications. Typically, such methods are employed to detect the presence or amount of an analyte in a sample, and/or the rate of binding of the analyte to the anti-analyte. Typical analyte-anti-analyte pairs include complementary strands of nucleic acids, antigen-antibody pairs, and receptor-ligand pairs, where the analyte can be either member of the pair, and the anti-analyte molecule, the opposite member.

Co-owned U.S. Pat. No. 5,804,453 (the '453 patent), which is incorporated herein by reference, discloses a fiber-optic interferometer assay device designed to detect analyte binding to a fiber-optic end surface. Analyte detection is based on a change in the thickness and the density at the end surface of the optical fiber resulting from the binding of analyte molecules to the surface, with greater amount of analyte producing a greater thickness and the density-related change in the interference signal. The change in interference signal is due to a phase shift between light reflected from the end of the fiber and from the binding layer carried on the fiber end, as illustrated particularly in FIGS. 7a and 7b of the '453 patent. The device is simple to operate and provides a rapid assay method for analyte detection.

Although interferometry-based assays have many advantages, including ease of use, low cost, and the ability to characterize binding kinetics, such techniques can be insufficiently sensitive to detect analytes that are present in extremely low concentrations (e.g., picomolar) or very low molecular weight (e.g., less than about 500 daltons, less than about 400 daltons, less than about 300 daltons, less than about 200 daltons, etc.) analytes. Thus there remains a need for improved interferometry-based assays to detect low concentration and/or low molecular weight analytes. The present invention provides for these and other advantages as described below.

### SUMMARY

In a first embodiment, a method is provided for detecting an analyte in a sample comprising providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises an analyte-binding molecule capable of specifically binding an analyte; performing an enzyme-linked assay for detecting binding of said analyte to said analyte binding molecule comprising: exposing said optical assembly to a sample comprising said analyte; binding an enzyme to said optical assembly wherein, the amount of bound enzyme is proportional to or inversely proportional to the concentration of analyte in said sample, and wherein said enzyme catalyzes a reaction to produce a precipitated substrate, and exposing said optical element to a precipitating substrate wherein upon binding of said precipitating substrate to said enzyme, said precipitated substrate is produced; and detecting a change in thickness at said first reflective surface arising from said precipitated substrate, thereby detecting said analyte in said sample.

In a first aspect of the first embodiment the amount of bound enzyme is proportional to the concentration of analyte in said sample.

In a second aspect of the first embodiment the amount of bound enzyme is inversely proportional to the concentration of analyte in said sample.

In a third aspect of the first embodiment detecting a change in thickness at said first reflective surface comprises detecting a shift in an interference pattern produced by reflection from the first reflective surface and the second reflective surface.

In a fourth aspect of the first embodiment said enzyme is horse radish peroxidase or alkaline phosphatase.

In a fifth aspect of the first embodiment said precipitating substrate comprises a component selected from the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

In a sixth aspect of the first embodiment said enzyme-linked assay comprises a competition assay.

In a seventh aspect of the first embodiment said enzyme-linked assay comprises an enzyme-linked bridging assay.

In a second embodiment a method is provided for detecting an analyte in a sample comprising: providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises a first analyte-binding molecule capable of specifically binding an analyte; exposing said optical element to a sample comprising said analyte; exposing said optical element to a second analyte binding molecule capable of specifically binding said analyte, and comprising a portion capable of specifically binding to an enzyme-carrying binding molecule, wherein said enzyme catalyzes a reaction to produce a precipitated substrate; exposing said optical element to said enzyme-carrying binding molecule exposing said optical element to a precipitating substrate, wherein upon binding of said precipitating substrate to said enzyme, said precipitated substrate is produced; and detecting a change in a thickness at said first reflective surface arising from said precipitated substrate, thereby detecting said analyte in said sample.

In a first aspect of the second embodiment said enzyme horse radish peroxidase or alkaline phosphatase.

In a second aspect of the second embodiment said precipitating substrate comprises a component selected from the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

In a third aspect of the second embodiment said analyte comprises a first antibody.

In a fourth aspect of the second embodiment said first analyte binding molecule comprises a second antibody.

In a third embodiment, an optical assembly is provided, said assembly comprising an optical element adapted for coupling to a light source via a fiber, said optical element comprising a transparent material, a first reflective surface and a second reflecting surface separated from said first surface by a distance, d, wherein said first reflective surface comprises: a first analyte-binding molecule capable of specifically binding an analyte; said analyte; a second analyte binding molecule capable of specifically binding said analyte; and a precipitated substrate.

In a first aspect of the third embodiment said reflective surface further comprises streptavidin.

In a second aspect of the third embodiment said analyte comprises an antibody.

In a third aspect of the third embodiment said precipitated substrate precipitates upon contact with horse radish peroxidase.

In a fourth embodiment, a kit for carrying out the methods of the invention is provided comprising: providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises a first member of a first binding pair; a first portion of a second member of said first binding pair; a second portion of a first member of a second binding pair; a third portion of a second member of said second binding pair wherein said second member of said second binding pair carries an enzyme wherein said enzyme catalyzes a reaction to produce a precipitated substrate upon binding with a precipitating substrate; packaging; and instructions for use.

In a first aspect of the fourth embodiment, the kit further comprises a fourth portion of said precipitating substrate.

In a second aspect of the fourth embodiment said first member of said first binding pair is streptavidin.

In a third aspect of the fourth embodiment said second member of said first binding pair is biotin.

In a fourth aspect of the fourth embodiment said first member of said second binding pair is fluorescein and said second member of said second binding pair is anti-fluorescein antibody.

In a fifth aspect of the fourth embodiment said precipitating substrate comprises a first component selected form the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

In a sixth aspect of the fourth embodiment said enzyme is horse radish peroxidase or alkaline phosphatase.

### BRIEF DESCRIPTION OF THE DRAWINGS

FIG. 1 illustrates an optical assembly according to one embodiment.

FIG. 2 illustrates a removably-carried optical assembly according to one embodiment.

FIG. 3 illustrates an optical assembly and enzyme-linked bridging assay according to one embodiment.

FIG. 4 illustrates signal modulation using various precipitating substrates.

FIG. 5 illustrates signal from varying concentrations of human IgG during calibration using protein A sensor.

FIG. 6 is a first dose response curve measuring anti-therapeutic antibodies in samples that include the therapeutic.

FIG. 7 are binding curves, measuring anti-therapeutic antibodies where some samples include the therapeutic in various sample matrix conditions, sample diluents and diluted human serum.

### DETAILED DESCRIPTION OF THE INVENTION

### Definitions

Terms used in the claims and specification are to be construed in accordance with their usual meaning as understood by one skilled in the art and as defined as set forth below. Numeric ranges recited in the claims and specification are to be construed as including the limits bounding the recited ranges.

An “analyte-binding molecule” refers to any molecule capable of participating in a specific binding reaction with an analyte molecule. Examples include but are not limited to, e.g., antibody-antigen binding reactions, drug-receptor binding interactions, antibody-binding molecules (such as protein A, protein G, and protein L), and nucleic acid hybridization reactions.

A “specific binding reaction” refers to a binding reaction that is saturable, usually reversible, and that can be competed with an excess of one of the reactants. Specific binding reactions are characterized by complementarity of shape, charge, and other binding determinants as between the participants in the specific binding reaction.

An “antibody” refers to an immunoglobulin molecule having two heavy chains and two light chains prepared by any method known in the art or later developed and includes polyclonal antibodies such as those produced by inoculating a mammal such as a goat, mouse, rabbit, etc. with an immunogen, as well as monoclonal antibodies produced using the well-known Kohler Milstein hybridoma fusion technique. The term includes antibodies produced using genetic engineering methods such as those employing, e.g., SCID mice reconstituted with human immunoglobulin genes, as well as antibodies that have been humanized using art-known resurfacing techniques.

An “antibody fragment” refers to a fragment of an antibody molecule produced by chemical cleavage or genetic engineering techniques, as well as to single chain variable fragments (SCFvs) such as those produced using combinatorial genetic libraries and phage display technologies. Antibody fragments used in accordance with the present invention usually retain the ability to bind their cognate antigen and so include variable sequences and antigen combining sites.

An “enzyme-linked bridging assay” is an assay that uses a surface-immobilized analyte binding molecule to capture an analyte of interest. The captured analyte binds to a second analyte binding molecule labeled (directly or indirectly) with an enzyme. The analyte thus acts as to “bridge” two analyte binding molecules, one used to capture the analyte, the other to provide a label for detecting the captured analyte. A sandwich enzyme-linked immunosorbent assay (“ELISA”) is one example of an enzyme-linked bridging assay.

A “precipitating substrate” refers to a compound that forms a precipitated substrate when it comes into contact with certain enzymes. When used with an enzyme-linked bridging assay, the precipitating substrate is preferably added to the assay mix after the analyte and second analyte binding molecule have been immobilized. An exemplary enzyme useful for enzyme-linked bridging assays is horse radish peroxidase (HRP) which catalyzes precipitation of 3,3′-diaminobenzidine tetrahydrochloride (DAB), 3,3′,5,5′-tetramethylbenzidine (TMB) and chloronaphthol (CN). Another exemplary enzyme is alkaline phosphatase which catalyzes precipitation of nitro-blue tetrazolium chloride (NBT) and 5-bromo-4-chloro-3′-indolyphosphate p-toluidine salt (BCIP). Additional examples include substrates used with Western blotting. Examples of enzymes and corresponding precipitating substrates include, but are not limited to:

| Enzyme | Precipitating Substrates |
|---|---|
| Horse radish peroxidase (HRP) | LumiGlo peroxidase chemiluminescent |
| | substrate (KPL, 54-61-00) |
| Horse radish peroxidase (HRP) | LumiGlo Reserve chemiluminescent |
| | substrate (KPL, 54-71-00) |
| Horse radish peroxidase (HRP) | StableDAB peroxidase substrate |
| | (KPL, 54-11-00) (DAB is 3,3′- |
| | diaminobenzidine tetrahydrochloride ) |
| Horse radish peroxidase (HRP) | HistoMark Trueblue peroxidase |
| | substrate (KPL, 54-78-00) |
| Horse radish peroxidase (HRP) | HistoMark Black peroxidase substrate |
| | (KPL, 54-75-00) |
| Horse radish peroxidase (HRP) | HistoMark Orange peroxidase substrate |
| | (KPL, 54-74-00) |
| Horse radish peroxidase (HRP) | TrueBlue peroxidase substrate (KPL, |
| | 71-00-64) |
| Horse radish peroxidase (HRP) | TMB 1-component membrane |
| | peroxidase substrate (50-77-18) |
| Horse radish peroxidase (HRP) | DAB substrate (Thermo, 1855900) |
| Horse radish peroxidase (HRP) | CN/DAB substrate (Pierce 34000) |
| Horse radish peroxidase (HRP) | Metal DAB (Thermo, 1856090) |
| Alkaline phosphatase (ALP, | NBT-BCIP substrate (Pierce, 34042) |
| Sigma-Aldrich A5719) | |

A “small molecule” refers to an organic compound having a molecular weight less than about 500 daltons.

It must be noted that, as used in the specification and the appended claims, the singular forms “a,” “an,” and “the” include plural referents unless the context clearly dictates otherwise.

All publications described herein are incorporated by reference in their entirety for all purposes.

Steps of methods described in the specification and appended claims can be performed in any order that results in an operative assay.

Advantages and Utility

Briefly, and as described in more detail below, provided are assemblies, kits and methods that are especially useful for carrying out fiber-based interferometry based assays using precipitating substrates to improve signals arising from binding of low molecular weight analytes, and/or analytes present at very low concentrations.

One way to amplify signal generated with Bio-Layer Interferometry (BLI) technology is to increase the number of binding molecules and/or change the property of the binding molecule to become more responsive to the optical measuring system. BLI is an optical analytical technique that analyzes the interference pattern of light reflected from two surfaces; a layer of immobilized molecules on the biosensor tip and an internal reference layer. A change in the number and/or property of the molecule bound to the biosensor tip causes a measurable change in the interference pattern. Only those molecules binding to or dissociating from the biosensor change the interference pattern and generate a response profile on the BLI system. Unbound molecules and changes in the refractive index of the surrounding medium produce no measurable effect on the pattern.

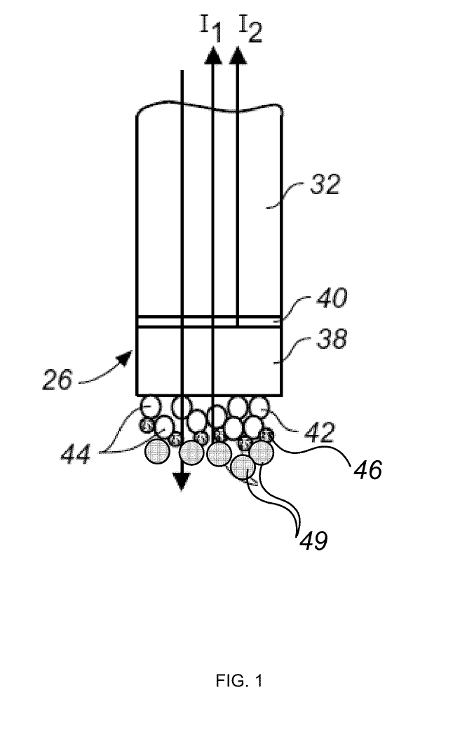

FIG. 1 shows, in schematic view, an optical assembly 26 adapted for coupling to a light source via optical waveguide or fiber 32. When used as a biosensor in a fiber-optic assay apparatus based on phase-shift interferometry (as illustrated FIG. 1 of co-owned U.S. Pat. No. 7,394,547), an interference pattern is generated by rays I₁ and I₂. Changes in that interference pattern are used to detect binding of analytes 46 to analyte-binding molecules 44.

Suitable optical fiber and coupling components are detailed in the above-cited '453 patent. One exemplary coupler is commercially available from many vendors including Ocean Optics (Dunedin, Fla.).

In the embodiment shown in FIG. 1, the optical assembly 26 is fixedly attached to an adjoining portion of the distal end region of an optical fiber 32, although disposable fiber optic tips as described in co-owned U.S. Pat. No. 7,319,525 (which is hereby incorporated by reference in its entirety for all purposes) are expressly contemplated to be within the scope of the present invention. As shown, the assembly 26 includes a transparent optical element 38 having first and second reflecting surfaces 42, 40 formed on its lower (distal) and upper (proximal) end faces, respectively. In one embodiment of the invention, the thickness “d” of the optical element between its distal and proximal surfaces, i.e., between the two reflecting surfaces, is at least 50 nm, and preferably at least 100 nm. An exemplary thickness is between about 100-5,000 nm, preferably 400-1,000 nm. The first reflecting surface 42 comprises components of an enzyme-linked bridging assay which includes analyte-binding molecules, such as molecules 44, which are capable of binding analyte molecules 46 specifically and with high affinity. That is, the analyte and anti-analyte molecules are opposite members of a binding pair of the type described above, which can include, without limitations, antigen-antibody pairs and receptor-binding agent pairs. A precipitating substrate accumulates in the vicinity of the bound analyte molecules having reacted with an enzyme bound to a second analyte binding molecule. Element 49 represents the second analyte binding molecule and the accumulated precipitating substrate.

The index of refraction of the optical element is preferably similar to that of the first reflecting surface, so that reflection from the lower distal end of the end optical assembly occurs predominantly from the layer that includes the analyte-binding molecules and precipitating substrate, rather than from the interface between the optical element and the layer that includes the analyte-binding molecules and precipitating substrate. Similarly, as analyte molecules and precipitating substrate bind to the lower layer of the optical assembly, light reflection form the lower end of the assembly occurs predominantly from the layer formed by the analyte-binding molecules, bound analyte and precipitating substrate, rather than from the interface region. One exemplary material forming the optical element is SiO₂, e.g., a high-quality glass having an index of refraction of about 1.4-1.5. The optical element can also be formed of a transparent polymer, such as polystyrene or polyethylene, having an index of refraction preferably in the 1.3-1.8 range.

The second reflecting surface in the optical assembly is formed as a layer of transparent material having an index of refraction that is substantially higher than that of the optical element, such that this layer functions to reflect a portion of the light directed onto the optical assembly. Preferably, the second layer has a refractive index greater than 1.8. One exemplary material for the second layer is Ta₂O₅ with refractive index equal to 2.1. The layer is typically formed on the optical element by a conventional vapor deposition coating or layering process, to a layer thickness of less than 50 nm, typically between 5 and 30 nm.

The thickness of the first (analyte-binding) layer is designed to optimize the overall sensitivity based on specific hardware and optical components. The present invention uses conventional immobilization chemistries to attach an enzyme-linked bridging assay to the lower surface of the optical element and in turn to attach a precipitating substrate prior to the detection step of the enzyme-linked bridging assay. In one embodiment, the enzyme-linked bridging assay, the capture molecule is an antibody. Alternatively an antigen is the capture molecule if the antibody is the analyte to be detected.

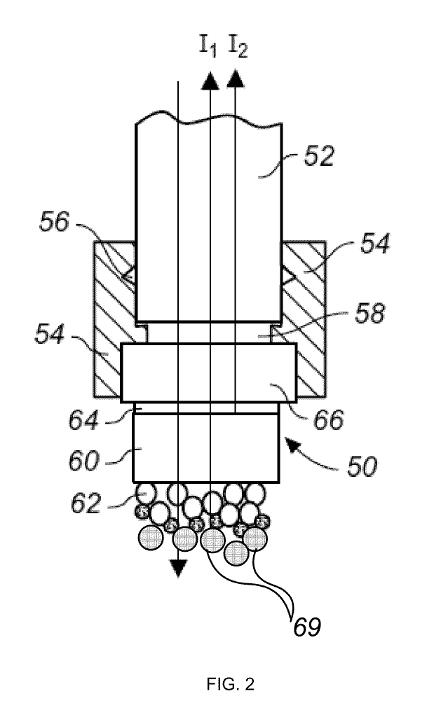

FIG. 2 shows an optical assembly 50 that is removably carried on the distal end of an optical fiber 52 in an assay apparatus. The optical element includes a plurality of flexible gripping arms, such as arms 54, that are designed to slide over the end of the fiber and grip the fiber by engagement of an annular rim or detente 56 on the fiber with complementary-shaped recesses formed in the arms, as shown. This attachment serves to position the optical assembly on the fiber to provide a gap 58 between the distal end of the fiber and the confronting (upper) face of the assembly, of less than 100 nm or greater than 2 μm. In one embodiment, the gap 58 is an air gap. In an alternative embodiment, the gap 58 is filled with a gel. With a gap 58 of greater than about 100 nm, but less that 2 μm, internal reflection from the upper surface of the optical assembly can contribute significantly to undesirable fringes that can adversely impact the detection accuracy.

With continued reference to FIG. 2, the optical assembly includes a first optical element 60 similar to optical element 38 described above, and having first and second reflective layers 62, 64, respectively, corresponding to above-described reflective layers 42, 40, respectively. Reflective layer 62 comprises an enzyme-linked bridging assay including second analyte binding molecule and precipitating substrate 69 at the distal surface of element 60. The assembly further includes a second optical element 66 whose thickness is preferably greater than 100 nm, typically at least 200 nm, and whose index of refraction is similar to that of first optical element 60. Preferably, the two optical elements are constructed of the same glass or a polymeric material having an index of refraction of between about 1.4 and 1.6. Layer 64, which is formed of a high index of refraction material, and has a thickness preferably less than about 30 nm, is sandwiched between the 2 optical elements as shown.

In operation, the optical assembly is placed over the distal fiber end and snapped into place on the fiber. The lower surface of the assembly is then exposed to a sample of analyte, under conditions that favor binding of sample analyte to the analyte-binding molecules comprising reflective layer 62. As analyte molecules bind to this layer, the thickness of the layer increases, increasing the distance “d” between reflective surfaces 62 and 64. This produces a shift in the interference pattern produced by reflection from the two layers. This shift, which can be measured as a shift in extrema (i.e., interference fringes) or shift in the wavelength of the interference pattern at a fixed position, etc., in turn, is used to determine the change in thickness at the lower (distal-most) reflecting layer. After use, the optical assembly can be removed and discarded, and replaced with fresh element for a new assay, for assaying the same or a different analyte.

Several features of the current approach should be noted. Measurements can be carried out using extremely small sample volumes (e.g., nL). Measurements can be carried out in vivo or in vitro. Sensitivity is increased by using a precipitating substrate to deposit a precipitate on the sensor tip in an amount directly or inversely (in the case of competition assays) proportional to bound analyte. This effectively amplifies the signal, by augmenting the amount of material accumulated on the BLI sensor tip. Optionally, the optical properties (e.g., color, refractive index, etc.) of the precipitate can further enhance BLI signal generation.

It is not necessary to remove unprecipitated molecules from the solution for detecting. Unbound molecules and changes in the refractive index of the surrounding medium do not affect the interference pattern.

### Enzyme-Linked BLI Assays

Enzyme-Linked Bridging Assays

Enzyme-linked bridging assays are used in combination with a precipitating substrate. In some embodiments the bridging assay uses a first binding pair (e.g., avidin, streptavidin, biotin, a hapten) to attach an analyte capture molecule to the BLI sensor tip. Analyte is captured by the analyte capture molecule. Captured analyte binds a second molecule associated (directly or indirectly) with an enzyme that reacts with a precipitating substrate to generate a precipitate. Any type of bridging enzyme-linked assay using a precipitating substrate known in the art can be adapted for use with the interferometry-based assays of the present invention as disclosed herein. Such adaptation merely requires modifying the analyte capture molecule so that it binds to the BLI sensor tip.

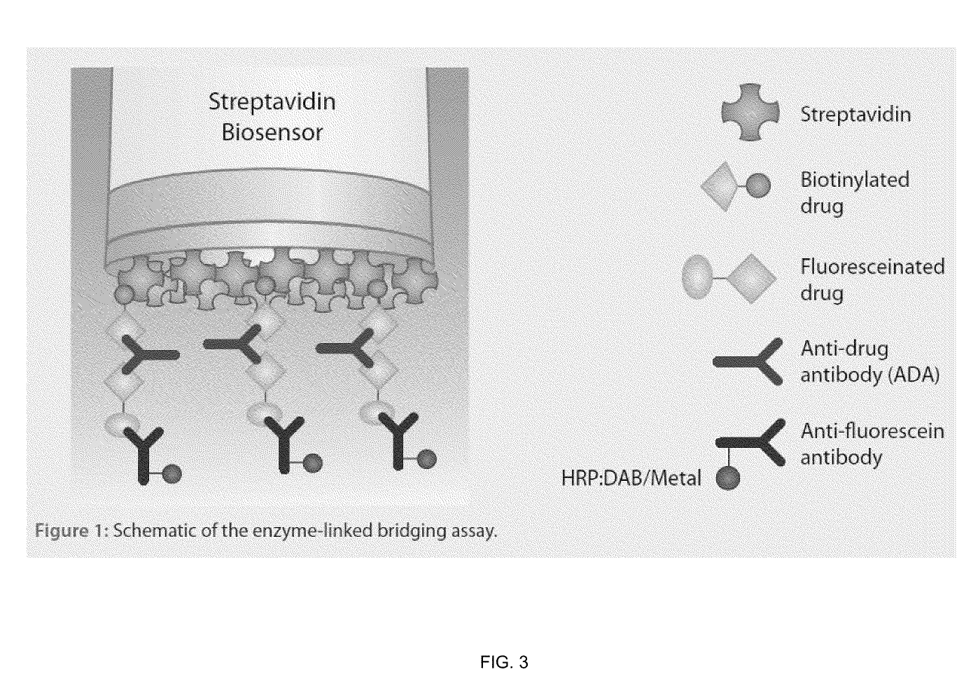

Multiple binding pairs can be used in the bridge. Referring to FIG. 3, an assay for detecting anti-drug antibodies (ADA) (also referred to as anti-therapeutic antibodies (ATA)), streptavidin is immobilized on the surface of the optical assembly. The drug is biotinylated and binds to the optical assembly through a biotin-streptavidin interaction (first binding pair). The ADA analyte is captured by the drug (second binding pair). Fluoresceinated drug binds the captured ADA (third binding pair) and an enzyme-linked (here, horseradish peroxidase) anti-fluorescein antibody binds to the fluorescein (fourth binding pair). In these embodiments, the same type of optical element (i.e., carrying one member of the binding pair) can be used in a wide variety of enzyme assays of the present invention so long as at least one other binding pair member carries an enzyme that acts on a precipitating substrate. For example the streptavidin-biotin binding pair can be adapted for use with many analytes and their anti-analyte binding partners.

Enzyme-Linked Competition Assays

In another embodiment, a competition assay is used in combination with a precipitating substrate. Techniques for competition assays are well-known in the art and are useful in the detection of small molecule analytes.

In one embodiment, the optical assembly is derivatized with a capture molecule (e.g., antibody to the analyte, receptor for the analyte, etc.). The derivatized optical assembly is incubated with an enzyme-labeled analyte wherein the enzyme catalyzes precipitation of the precipitating substrate and a sample that includes the analyte of interest (which is not labeled), under conditions that allow for competitive binding of the labeled and unlabeled analyte to the capture molecule to take place. After the reaction equilibrates, or after a predetermined time the optical assembly is exposed to the precipitating substrate. Precipitated substrate forms where the derivatized analyte remains bound to the optical assembly. Thus lower signal results from higher concentrations analyte present in the sample. Analyte can be quantified through use of a standard curve constructed using known concentrations of unlabeled analyte (as is standard in this art). In one embodiment, the derivatized optical assembly is first incubated with the enzyme-labeled analyte prior to incubating with the sample comprising the unlabeled analyte. In another embodiment, the derivatized optical assembly is first incubated with the sample comprising the unlabeled analyte before adding the enzyme-labeled analyte. In yet another variation, the derivatized optical assembly is exposed simultaneously to the enzyme-labeled analyte and the sample comprising the unlabeled analyte.

Precipitated substrate forms in an amount inversely proportional to the concentration of (unlabeled) analyte present in the sample.

The assays of the present invention can be used to detect an ADA as part of an immune response to a protein or antibody drug. These methods can be used with human, primate and other animal serum and plasma samples.

Advantages of this approach are numerous. Because the invention uses precipitating substrates to amplify binding signals, assay sensitivity is enhanced, allowing for better detection of analytes present at low concentrations, and/or low molecular weight analytes. The assays of the present invention retain these advantages even when used to detect analytes present in complex samples.

In one embodiment, the disclosed assays are useful for detecting antibodies to drugs in samples from patients. Patient samples include not only many biomolecules but also may include the drug recognized by the antibody analyte under investigation, or an immunologically cross-reacting drug metabolite. The assays of the present invention are able to accurately detect anti-drug antibody in such a complex sample.

### Kits of the Invention

Kits of the invention include a glass fiber adapted for coupling to a bio-layer interferometer, reagents and instructions for derivatizing the glass fiber with a first substrate, derivatizing assay components, capturing an analyte and derivatizing the analyte with a precipitating substrate.

In one embodiment, the kit includes reagents in multiple binding pairs such that the glass fiber is adapted to perform an enzyme-linked bridging assay with a precipitating substrate. A first reagent comprises a first member of a first binding pair to derivatize the glass fiber (e.g., streptavidin, avidin, etc.). In another embodiment, the kit can include a glass fiber that already has been derivatized with a first member of a first binding pair. A second reagent is the second member of the first binding pair (e.g., biotin) to derivatize the capture molecule such that it can be bound to the glass fiber via the first member of the binding pair. The first binding pair can also be any other binding pair. A third reagent in the kit is a first member of a second binding pair. This first member of the second binding pair is used to derivatize additional capture molecule (e.g., fluorescein). The fourth reagent comprises the second member of the second binding pair (e.g., anti-fluorescein) and an enzyme that reacts with the precipitating substrate to form a precipitate (e.g., horse radish peroxidase, alkaline phosphatase, etc.). The second binding pair can also be any other binding pair. The fifth reagent comprises the precipitating substrate. Examples of fifth reagents when the enzyme is horse radish peroxidase include compositions comprising 3,3′-diaminobenzidine tetrahydrochloride (DAB), 3,3′,5,5′-tetramethylbenzidine (TMB), and chloronaphthol (CN). Examples of fifth reagents when the enzyme is alkaline phosphatase) include nitro-blue tetrazolium chloride (NBT) and 5-bromo-4-chloro-3′-indolyphosphate p-toluidine salt (BCIP). Kits can include all of the above-described reagents, or any sub-combination, along with packaging and instructions for use.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but one should allow for some experimental error and deviation.

The practice of the present invention will employ, unless otherwise indicated, conventional methods of protein chemistry, biochemistry, and pharmacology, within the skill of the art. Such techniques are explained fully in the literature. See, e.g., T. E. Creighton, Proteins: Structures and Molecular Properties (W.H. Freeman and Company, 1993); D. L. Nelson and M. M. Cox, Lehninger: Principles of Biochemistry, Fourth Edition (W.H. Freeman and Company, 2005); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 5th Ed. (Springer Science and Business) Vols A and B (2007).

### Example 1

### Detecting Anti-Drug Antibodies

An assay was developed using a streptavidin biosensor to capture a biotin-drug/ADA/fluorescein drug/anti-fluorescein isothiocyanate (FITC) horseradish peroxidase (HRP) complex out of the treated sample mixture as shown by the diagram of FIG. 3. The method was used in combination with biolayer interferometry such as the Octet QK384/RED384 instrument available from FortéBio of Menlo Park, Calif.

Materials used were:
40 μL samples for each test
purified drug for immobilization and detection −1 mg needed for biotinylation and fluorescein labeling; 4 μg for each enzyme-linked assay
phosphate buffered saline (PBS)
96 well microplates (Greiner Bio-one C/N 655209)
EZ-Link Biotin-LC-LC-NHS (Thermo C/N 21343)
Dimethyl formamide (DMF) to dissolve Biotin-LC-LC-NHS and Fluorescein-NHS (Thermo C/N 20672)
PD-10 column desalting column (GE Healthcare C/N 17-0851-01)
Fluorescein-NHS (Thermo C/N 46410)
Peroxide Substrate Buffer (PSB) (Thermo C/N 1855910)
DAB/Metal Concentrate (10×) (Thermo C/N 1856090)
Rabbit anti-FITC:HRP (AbDSerotec C/N 4510-7864)

Samples, reagents and buffers were equilibrated at room temperature and mixed thoroughly prior to use. In one example, a minimum of 200 μL was placed in each well of a 96-well plate. In another example using a 384-well plate such as with Octet RED384 and Octet QK384 (both available from FortéBio of Menlo Park, Calif.), a minimum of 80 μL was used in each well.

For the 96-well plate, 40 μL of sample, 40 μL of biotin-drug/fluorescein-drug and 120 μL of immunogenicity reagent were combined. This provides a total of 200 μL. The plate was covered and incubated at room temperature for 1-2 hours (with agitation on an orbital shaker) or incubated 4 hours with no shaking.

For proceeding with the Octet QK/RED instrument and a 96-well plates or the Octet QK384/RED384 and a 384-well plate, 2 mLs each were prepared of a solution of anti-fluorescein-HRP antibody conjugate diluted 1:1000 in sample diluent (1 mg/mL BSA and 0.02% Tween 20 in PSB at pH 7.4)/ProClin300 (a preservative comprising 2-methyl-4-isothiazolin-3-one and 5-chloro-2-methyl-4-isothiazolin-3-one available from Sigma Aldrich) and a solution of metal/DAB substrate diluted 1:10 in PSB. After incubation 200 μL anti-fluorescein-HRP antibody conjugate was added. After three minutes incubation, 200 μL metal-DAB substrate was added. The incubation time between the anti-fluorescein-HRP addition and the metal-DAB substrate addition is flexible. The longer the incubation time, the more the signal is modulated.

For proceeding with the Octet QK384/RED384 instrument and a 96-well plate, 4 mLs each were prepared of a solution of anti-fluorescein-HRP antibody conjugate diluted 1:1000 in sample diluent/ProClin300 and a solution of metal/DAB substrate diluted 1:10 in Peroxide Substrate Buffer. After incubation, anti-fluorescein-HRP antibody conjugate was added followed by the metal-DAB substrate as for the Octet QK/RED instrument.

### Example 2

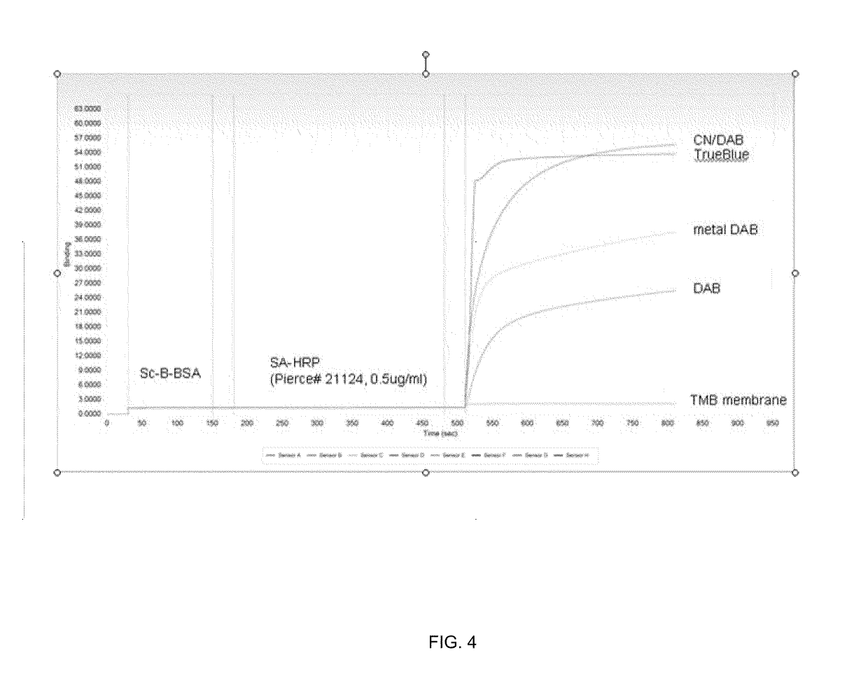

FIG. 4 illustrates the increase in signal using various precipitating substrates. Biotinylated streptavidin biosensors were reacted with 5 μg/mL streptavidin-HRP conjugate. The sensors were then washed with sample diluent. Sensors were placed in a well with the HRP substrates for 5 minutes and the generated signal was read with the Octet RED instrument. CN/DAB (Pierce 34000), HistoMark TrueBlue (KPL, 54-78-00), metal DAB (Thermo, 1856090), DAB (Thermo, 1855900) and TMB 1-component membrane peroxidase (50-77-18) were the tested HRP substrates, added at 500 seconds. The dramatic improvement in signal provided by the precipitating substrate is seen in how the signal increased upon addition of the precipitating substrate.

### Example 3

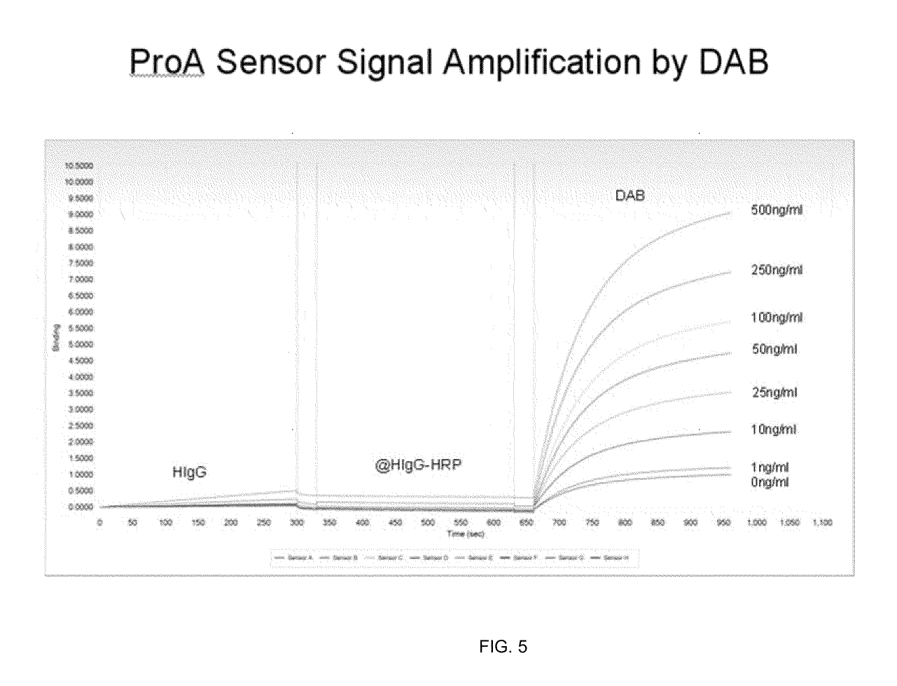

FIG. 5 illustrates signal from varying concentrations of human IgG during calibration using protein A sensor. The signals were greatly amplified by HRP enzyme and precipitating substrate DAB in the range of 1-500 ng/mL. Biosensors derivatized with protein A (as a capture molecule) were incubated with samples containing 1 ng/ml, 10 ng/ml, 25 ng/ml, 50 ng/ml, 100 ng/ml, 250 ng/ml and 500 ng/ml of human IgG (HIgG). They were next reacted with 5 μg/mL anti-human IgG-HRP conjugate. In this example, the sensors were then washed with sample diluent to remove unbound anti-human IgG-HRP. Sensors were placed in a well with DAB (HRP precipitating substrate) (at approximately 650 seconds) for 5 minutes and the generated signal was read with the Octet RED instrument. Here as well, the dramatic improvement in signal provided by the precipitating substrate is seen in how the signal increased upon addition of the precipitating substrate. Comparison of the signals arising from HIgG binding (time 0 to 300 seconds) to those arising from DAB precipitate (times 650 to 950 seconds) illustrates the dramatic amplification produced by precipitating substrate. Assuming IgG has a mass of approximately 150 kDa, the precipitating substrate amplification allows reliable detection of analyte at concentrations at least as low as 6.67 μM.

### Example 4

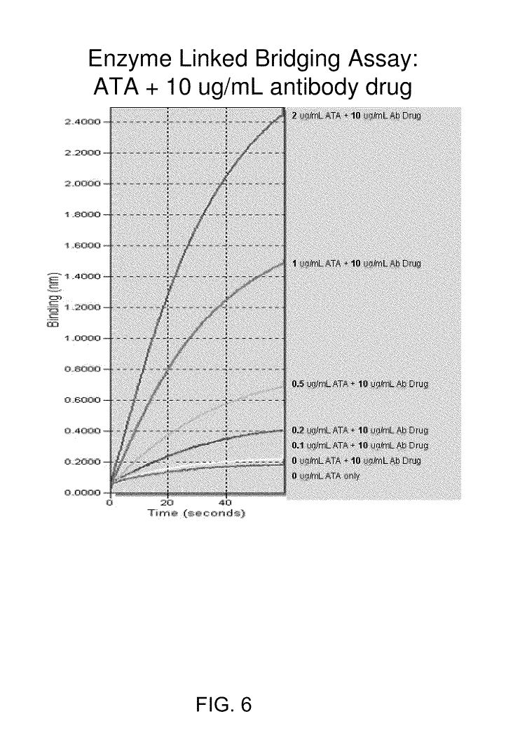

FIG. 6 is a dose-response curve made using procedure of Example 1. Concentration of an ADA to an antibody drug was determined in samples that also include the antibody drug to which the ADA was formed. FIG. 6 demonstrates that the assay detects down to 100 ng/mL in the presence of 10 m/mL free antibody drug. This demonstrates that the assay tolerates 100 fold molar excess of free antibody drug to ADA.

### Example 5

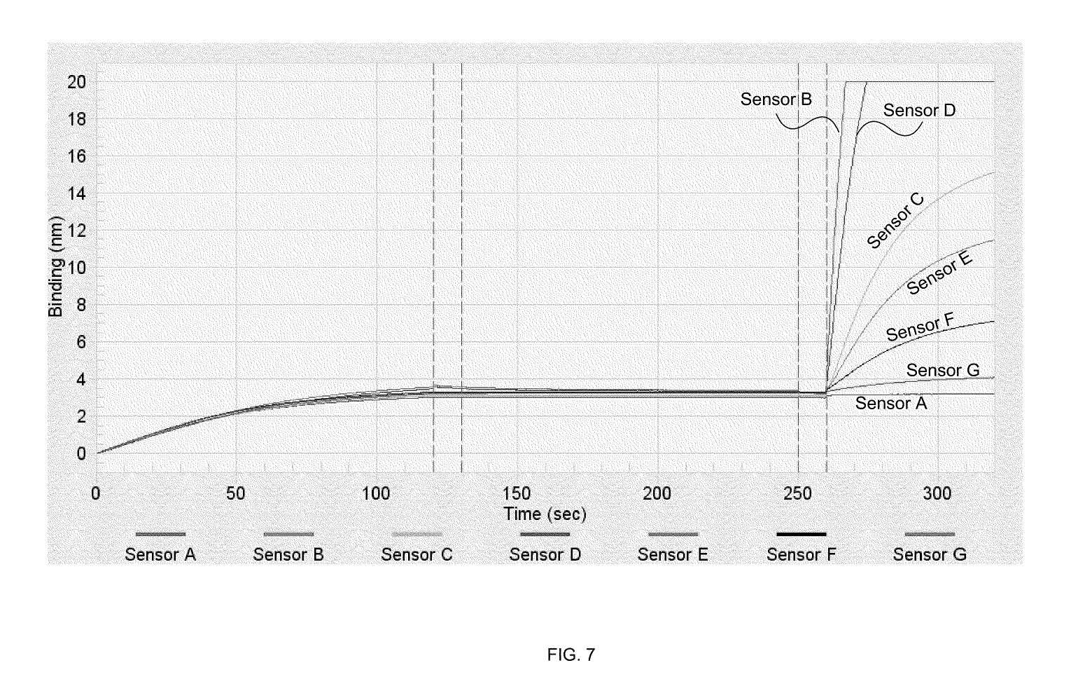

FIG. 7 illustrates detection of ADA in various sample matrices (SD and 20% serum) with and without the presence of 50 ug/L of free drug to which the ADA was formed. These conditions address the assay sensitivity and drug tolerance by detecting ADA with and without the presence of the free drug. In this example, this assay detected 1 ug/mL of ADA and tolerated up to 50 μg/mL of drug in both SD and 20% serum matrix. The experiment followed the procedure of Example 1. The derivatization of each sensor is shown in the chart below:

| Samples | Signal |
|---|---|
| A: serum ctrl | 0.2 |
| G: 1 ug/ml ADA + 50 ug/ml Drug in 20% serum | 0.8 |
| F: 1 ug/ml ADA + 50 ug/ml Drug in SD | 3.7 |
| B: 4 ug/ml ADA in SD | 38.3 |
| D: 4 ug/ml ADA in 20% serum | 28.0 |
| C: 1 ug/ml ADA in SD | 11.8 |
| E: 1 ug/ml ADA in 20% serum | 8.0 |

In alternate embodiments, the experiment of Example 5 uses a longer incubation times for sample incubation and/or HRP incubation. It is expected to improve the assay sensitivity with longer incubation times of ADA and/or HRP conjugate.

Because the assays of Examples 4 and 5 use derivatized drug to detect ADA, having a sample that includes the drug could have affected the assay's sensitivity. Examples 4 and 5 illustrate that the assays tolerate the presence of the drug in the sample when measuring for the ADA.

1. A method for detecting an analyte in a sample comprising:
providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises an analyte-binding molecule capable of specifically binding an analyte;
performing an enzyme-linked assay for detecting binding of said analyte to said analyte binding molecule comprising:
exposing said optical assembly to a sample comprising said analyte;
binding an enzyme to said optical assembly wherein, the amount of bound enzyme is proportional to or inversely proportional to the concentration of analyte in said sample, and wherein said enzyme catalyzes a reaction to produce a precipitated substrate, and exposing said optical element to a precipitating substrate wherein upon binding of said precipitating substrate to said enzyme, said precipitated substrate is produced; and
detecting a change in thickness at said first reflective surface arising from said precipitated substrate, thereby detecting said analyte in said sample.

2. The method of claim 1, wherein the amount of bound enzyme is proportional to the concentration of analyte in said sample.

3. The method of claim 1, wherein the amount of bound enzyme is inversely proportional to the concentration of analyte in said sample.

4. The method of claim 1, wherein detecting a change in thickness at said first reflective surface comprises detecting a shift in an interference pattern produced by reflection from the first reflective surface and the second reflective surface.

5. The method of claim 1, wherein said enzyme is horse radish peroxidase or alkaline phosphatase.

6. The method of claim 1, wherein said precipitating substrate comprises a component selected from the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

7. The method of claim 1 wherein said enzyme-linked assay comprises a competition assay.

8. The method of claim 1 wherein said enzyme-linked assay comprises an enzyme-linked bridging assay.

9. A method for detecting an analyte in a sample comprising:
providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises a first analyte-binding molecule capable of specifically binding an analyte;
exposing said optical element to a sample comprising said analyte;
exposing said optical element to a second analyte binding molecule capable of specifically binding said analyte, and comprising a portion capable of specifically binding to an enzyme-carrying binding molecule, wherein said enzyme catalyzes a reaction to produce a precipitated substrate;
exposing said optical element to said enzyme-carrying binding molecule
exposing said optical element to a precipitating substrate, wherein upon binding of said precipitating substrate to said enzyme, said precipitated substrate is produced; and
detecting a change in a thickness at said first reflective surface arising from said precipitated substrate, thereby detecting said analyte in said sample.

10. The method of claim 9 wherein said enzyme horse radish peroxidase or alkaline phosphatase.

11. The method of claim 9 wherein said precipitating substrate comprises a component selected from the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

12. The method of claim 9 wherein said analyte comprises a first antibody.

13. The method of claim 12 wherein said first analyte binding molecule comprises a second antibody.

14. An optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises:
a first analyte-binding molecule capable of specifically binding an analyte;
said analyte;
a second analyte binding molecule capable of specifically binding said analyte; and
a precipitated substrate.

15. The optical assembly of claim 14 wherein said reflective surface further comprises streptavidin.

16. The optical assembly of claim 14 wherein said analyte comprises an antibody.

17. The optical assembly of claim 14 wherein said precipitated substrate precipitates upon contact with horse radish peroxidase.

18. A kit comprising:
providing an optical assembly, said assembly comprising an optical element adapted for coupling to a light source via a fiber, said optical element comprising a transparent material, a first reflective surface and a second reflecting surface separated from said first surface by a distance, d, wherein said first reflective surface comprises a first member of a first binding pair;
a first portion of a second member of said first binding pair;
a second portion of a first member of a second binding pair;
a third portion of a second member of said second binding pair wherein said second member of said second binding pair carries an enzyme wherein said enzyme catalyzes a reaction to produce a precipitated substrate upon binding with a precipitating substrate;
packaging; and
instructions for use.

19. The kit of claim 18 further comprising a fourth portion of said precipitating substrate.

20. The kit of claim 18 wherein said first member of said first binding pair is streptavidin.

21. The kit of claim 18 wherein said second member of said first binding pair is biotin.

22. The kit of claim 18 wherein said first member of said second binding pair is fluorescein and said second member of said second binding pair is anti-fluorescein antibody.

23. The kit of claim 19 wherein said precipitating substrate comprises a first component selected form the group consisting of 3,3′-diaminobenzidine tetrahydrochloride (DAB), 3,3′,5,5′-tetramethylbenzidine (TMB), chloronaphthol (CN), nitro-blue tetrazolium chloride (NBT) and 5-bromo-4-chloro-3′-indolyphosphate p-toluidine salt (BCIP).

24. The kit of claim 18 wherein said enzyme is horse radish peroxidase or alkaline phosphatase.
